SEQUENCE LISTING (8 sequences)

SEQ ID NO: 1 - protein, 782 aa, synthetic construct
  source 1..782: note = pgp96
DDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEKDEL

SEQ ID NO: 2 - protein, 778 aa, synthetic construct
  source 1..778: note = rgp96
DDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAE

SEQ ID NO: 3 - DNA, 2334 bp, synthetic construct
  source 1..2334: mol_type = other DNA; note = nucleotide sequence encoding rgp96
gacgatgaagttgatgtggatggtacagtagaagaggatctgggtaaaagtagagaaggatcaaggacggatgatgaagtagtacagagagaggaagaagctattcagttggatggattaaatgcatcacaaataagagaacttagagagaagtcggaaaagtttgccttccaagccgaagttaacagaatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcgtcttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcccagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaggagacagcagaagacacaacagaagacacagagcaagacgaagatgaagaaatggatgtgggaacagatgaagaagaagaaacagcaaaggaatctacagctgaa

SEQ ID NO: 4 - protein, 790 aa, synthetic construct
  source 1..790: note = gp96-plus
GGGGSCGGGGSMDDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAE

SEQ ID NO: 5 - DNA, 2370 bp, synthetic construct
  source 1..2370: mol_type = other DNA; note = nucleotide sequence encoding gp96-plus
ggtggtggtggttcctgcggtggtggtggttccatggacgatgaagttgatgtggatggtacagtagaagaggatctgggtaaaagtagagaaggatcaaggacggatgatgaagtagtacagagagaggaagaagctattcagttggatggattaaatgcatcacaaataagagaacttagagagaagtcggaaaagtttgccttccaagccgaagttaacagaatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcgtcttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcccagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaggagacagcagaagacacaacagaagacacagagcaagacgaagatgaagaaatggatgtgggaacagatgaagaagaagaaacagcaaaggaatctacagctgaa

SEQ ID NO: 6 - protein, 11 aa, synthetic construct
  source 1..11: note = linker
GGGGSCGGGGS

SEQ ID NO: 7 - DNA, 25 bp, synthetic construct
  source 1..25: mol_type = other DNA; note = primer F1
ggaattcatggacgatgaagttgat

SEQ ID NO: 8 - DNA, 29 bp, synthetic construct
  source 1..29: mol_type = other DNA; note = primer R1
gctctagactattagaattcatctttttc

(19) United States
(12) Patent Application Publication
(10) Pub. No.: US 2025/0368702 A1
(43) Pub. Date: Dec. 4, 2025

(54) GP96 AND USE THEREOF IN TREATING AMYOTROPHIC LATERAL SCLEROSIS

(71) Applicant: FOSHAN HEAT SHOCK BIOTECH CO., LTD., Foshan (CN)

(72) Inventors: Songdong Meng, Foshan (CN); Ying Ju, Foshan (CN); Xin Li, Foshan (CN); Yuxiu Xu, Foshan (CN); Fang Cheng, Foshan (CN); Zihao Wang, Foshan (CN)

(21) Appl. No.: 18/998,401

(22) PCT Filed: Nov. 21, 2022
(86) PCT No.: PCT/CN2022/133209

§ 371 Date: Jan. 24, 2025

(30) Foreign Application Priority Data
Jul. 28, 2022 (CN) 202210898983.X

(51) Int. Cl. C07K 14/47; A61K 38/00; A61P 25/28

(52) U.S. Cl. CPC C07K 14/47; A61P 25/28; A61K 38/00; C07K 2319/00

(57) ABSTRACT

The present invention relates to the field of disease treatment. In particular, the present invention provides a gp96 protein and use of a gp96 protein-constructed fusion protein in treating amyotrophic lateral sclerosis. In addition, the present invention further relates to a pharmaceutical composition that can be used for treating one or more of the symptoms of amyotrophic lateral sclerosis, comprising the gp96 protein or the gp96 protein-constructed fusion protein of the present invention.

### TECHNICAL FIELD

The present application relates to the field of disease treatment. Specifically, the present application provides a use of gp96 protein and fusion protein constructed from gp96 protein for treating amyotrophic lateral sclerosis. In addition, the present application also relates to a pharmaceutical composition capable of being used to treat one or more symptoms of amyotrophic lateral sclerosis, which comprises the gp96 protein or the fusion protein constructed from the gp96 protein of the present invention.

### BACKGROUND ART

Amyotrophic lateral sclerosis (ALS), commonly known as “motor neuron disease”, is caused by progressive degeneration of motor nerve cells, mainly affecting motor neurons in the cortex, brainstem and spinal cord, leading to gradual weakness and atrophy of muscles in the limbs, trunk, chest and abdomen, as well as decreased speech, swallowing and respiratory functions, until respiratory failure and death. The cause of amyotrophic lateral sclerosis is still unknown. 20% of cases may be related to genetic and gene defects. In addition, some environmental factors, such as heavy metal poisoning, may cause motor neuron damage, but the specific pathogenic mechanism is still unclear. The incidence of ALS is very low, but it poses a great threat to the patient's living quality and life. The current drugs for treating ALS are edaravone (trade name Radicava) and Riluzole. According to the ALS Association, about 5,000 people are diagnosed each year, with an average life expectancy of two to five years, so patients are in urgent need of transformative treatment options.

Studies have shown that motor nerve damage and axonal lesions mediated by specific genetic backgrounds, reactive oxygen species and oxidative stress, neuroinflammation and autoimmune responses, Treg dysfunction and reduced level thereof, motor neuron mitochondrial dysfunction, metabolic disorder and dysfunction of motor neuron, protein denaturation, etc. may be key driving factors of ALS progression and neurodegenerative diseases.

### CONTENTS OF THE APPLICATION

Heat shock protein (HSP) is a type of protein that is highly conserved in biological evolution and widely present in prokaryotes and eukaryotes. Its main biological functions include: acting as a molecular chaperone, participating in the folding and assembly of newly synthesized proteins; binding to other peptides or proteins in a cell, especially denatured proteins, participating in anti-damage, repair and heat tolerance process of cells; participating in protein hydrolysis process; binding to antigenic peptides, processing and presenting tumor antigens and maintaining cellular homeostasis; as well as having a certain regulatory effect on cell growth, development, differentiation and death. Heat shock protein gp96 belongs to a family of heat shock proteins and has significant biological activity.

After extensive research, the inventors of the present application found that gp96 protein can be effectively used for the treatment of amyotrophic lateral sclerosis and has important application value in treating amyotrophic lateral sclerosis or alleviating the symptoms of amyotrophic lateral sclerosis.

In addition, the inventors of the present application obtained through research a fusion protein constructed from gp96 protein, which has improved therapeutic activity against amyotrophic lateral sclerosis in comparison with gp96 protein.

### Therapeutic Use

Therefore, in one aspect, the present application provides a use of a gp96 protein or variant thereof or a fusion protein in the manufacture of a medicament for preventing and/or treating amyotrophic lateral sclerosis in a subject;
wherein, the variant has a sequence identity of at least 90%, such as at least 95%, at least 96%, at least 97%, at least 98%, at least 99%, or, has a substitution (preferably conservative substitution), addition or deletion of one or several (e.g., 1, 2, 3, 4, 5, 6, 7, 8 or 9) amino acids, as compared to the gp96 protein, and retains the function of the gp96 protein;
the fusion protein comprises the gp96 protein or variant thereof, and an additional peptide connected to the gp96 protein or variant thereof.

In certain embodiments, the additional peptide is connected to the N-terminal and/or C-terminal of the gp96 protein or variant thereof, optionally via a linker (e.g., a peptide linker).

In certain embodiments, the additional peptide is connected to the N-terminal of the gp96 protein or variant thereof.

In some embodiments, the additional peptide is a flexible peptide.

In some embodiments, the additional peptide comprises one or more glycine (G).

In some embodiments, the additional peptide has a structure as set forth in (GGGGS)ₙ₁C(GGGGS)ₙ₂, wherein the n1 and n2 are each independently selected from: 0, 1, 2, 3, 4, 5, 6, 7, 8, 9, 10. In some embodiments, the n1 and n2 are not 0 at the same time.

In some embodiments, the additional peptide has an amino acid sequence as set forth in SEQ ID NO: 6.

It is known to those skilled in the art that during the translation of mRNA, due to the action of the start codon, the first position of the generated polypeptide chain is often an amino acid encoded by the start codon (e.g., methionine (M)). Therefore, the gp96 protein or variant thereof or the fusion protein of the present invention not only encompasses an amino acid sequence that does not comprise an amino acid encoded by a start codon (e.g., methionine) at its N-terminal, but also encompasses an amino acid sequence that comprises an amino acid encoded by a start codon (e.g., methionine) at its N-terminal.

In certain embodiments, the gp96 protein comprises or consists of an amino acid sequence as set forth in SEQ ID NO: 1 or 2. The sequence shown here does not comprise a methionine encoded by a start codon at its N-terminal. It is understood by those skilled in the art that the gp96 protein may also comprise or consist of the above amino acid sequence that comprises a methionine encoded by a start codon at its N-terminal.

In certain embodiments, the gp96 protein is produced by a genetic engineering method (recombinant technology). In certain embodiments, the gp96 protein is extracted from a natural biological sample. In certain embodiments, the gp96 protein is extracted from an animal ex vivo placental tissue. In certain embodiments, the gp96 protein is extracted from a human ex vivo placental tissue. In certain embodiments, the gp96 protein is extracted from a mouse ex vivo placental tissue.

In certain embodiments, the fusion protein comprises or consists of an amino acid sequence as set forth in SEQ ID NO: 4. The sequence shown here does not comprise a methionine encoded by a start codon at its N-terminal. Those skilled in the art understand that the fusion protein may also comprise or consist of the above amino acid sequence that comprises a methionine encoded by a start codon at its N-terminal.

In certain embodiments, the gp96 protein or variant thereof or the fusion protein may also comprise an additional protein tag, a targeting moiety or any combination thereof.

Herein, the protein tag is well known in the art, examples of which include but are not limited to His, Flag, GST, MBP, HA, Myc, GFP or biotin, and those skilled in the art know how to select a suitable protein tag according to the desired purpose (e.g., purification, detection or tracing).

Herein, the term “targeting moiety” refers to a domain that can guide the gp96 protein or variant thereof or the fusion protein of the present invention to a desired location, which may be a specific tissue, a specific cell, or even a specific intracellular location (e.g., a nucleus, a ribosome, an endoplasmic reticulum, a lysosome or a peroxisome). Those skilled in the art know how to design a corresponding targeting moiety based on the characteristics of the desired location. In certain embodiments, the targeting moiety comprises a ligand, a receptor or an antibody or binding domain thereof.

In certain embodiments, the medicament is used for one or more of the following:
(1) inducing a regulatory T cell;
(2) inhibiting the generation of a Th17 cell;
(3) inducing an increase in the number of Th2 cell;
(4) inhibiting the generation of a Th1 cell;
(5) reducing reactive oxygen species and oxidative stress in a motor neuron;
(6) reducing the expression of SOD1;
(7) restoring the function of dysfunctional mitochondria in a motor neuron;
(8) reducing denatured protein in a cell;
(9) reducing creatine kinase level and/or inhibiting creatine kinase activity, and upregulating creatine level;
(10) promoting the production of nerve growth factor;
(11) promoting the growth of a diseased motor nerve axon;
(12) improving axonal transport capacity.

In certain embodiments, the medicament is used for one or more of the following:
(1) inducing a regulatory T cell in a subject;
(2) inhibiting the generation of a Th17 cell in a subject;
(3) inducing an increase in the number of Th2 cell in a subject;
(4) inhibiting the generation of a Th1 cell in a subject;
(5) reducing reactive oxygen species and oxidative stress in a motor neuron in a subject;
(6) reducing the expression of SOD1 in a subject;
(7) restoring the function of dysfunctional mitochondria in a motor neuron in a subject;
(8) reducing denatured protein in a cell in a subject;
(9) reducing creatine kinase level and/or inhibiting creatine kinase activity, and upregulating creatine level in a subject;
(10) promoting the production of nerve growth factor in a subject;
(11) promoting the growth of a diseased motor nerve axon in a subject;
(12) improving axonal transport capacity in a subject.

In certain embodiments, the subject is a human or a mouse. In certain preferred embodiments, the subject is a human.

In certain embodiments, the regulatory T cell is a CD4+CD25+FOXP3+ regulatory T cell. In certain embodiments, the Th17 is a CD4+ T cell capable of producing IL-17 (interleukin 17). In certain embodiments, the Th1 is a CD4+ T cell capable of producing IFN-γ (interferon-γ), TNFβ (tumor necrosis factor β), granulocyte-macrophage colony-stimulating factor (GM-CSF), IL-2, lymphotoxin (LT). In certain embodiments, the Th2 is a CD4+ T cell capable of producing IL4, IL5, IL-9, IL-10 and IL-13.

In another aspect, the present application provides a method for preventing and/or treating amyotrophic lateral sclerosis, comprising: administering an effective amount of a gp96 protein or variant thereof or a fusion protein to a subject in need thereof; wherein the gp96 protein or variant thereof or the fusion protein is as defined above.

In certain embodiments, the method is used for one or more of the following:
(1) inducing a regulatory T cell;
(2) inhibiting the generation of a Th17 cell;
(3) inducing an increase in the number of Th2 cell;
(4) inhibiting the generation of a Th1 cell;
(5) reducing reactive oxygen species and oxidative stress in a motor neuron;
(6) reducing the expression of SOD1;
(7) restoring the function of dysfunctional mitochondria in a motor neuron;
(8) reducing denatured protein in a cell;
(9) reducing creatine kinase level and/or inhibiting creatine kinase activity, and upregulating creatine level;
(10) promoting the production of nerve growth factor;
(11) promoting the growth of a diseased motor nerve axon;
(12) improving axonal transport capacity.

In certain embodiments, the method is used for one or more of the following:
(1) inducing a regulatory T cell in a subject;
(2) inhibiting the generation of a Th17 cell in a subject;
(3) inducing an increase in the number of Th2 cell in a subject;
(4) inhibiting the generation of a Th1 cell in a subject;
(5) reducing reactive oxygen species and oxidative stress in a motor neuron in a subject;
(6) reducing the expression of SOD1 in a subject;
(7) restoring the function of dysfunctional mitochondria in a motor neuron in a subject;
(8) reducing denatured protein in a cell in a subject;
(9) reducing creatine kinase level and/or inhibiting creatine kinase activity, and upregulating creatine level in a subject;
(10) promoting the production of nerve growth factor in a subject;
(11) promoting the growth of a diseased motor nerve axon in a subject;
(12) improving axonal transport capacity in a subject.

In certain embodiments, the subject is a human or a mouse. In certain preferred embodiments, the subject is a human.

In certain embodiments, the regulatory T cell is a CD4+CD25+FOXP3+ regulatory T cell. In certain embodiments, the Th17 is a CD4+ T cell capable of producing IL-17 (interleukin 17). In certain embodiments, the Th1 is a CD4+ T cell capable of producing IFN-γ (interferon-gamma), TNFβ (tumor necrosis factor β), granulocyte-macrophage colony-stimulating factor (GM-CSF), IL-2, lymphotoxin (LT). In certain embodiments, the Th2 is a CD4+ T cell capable of producing IL4, IL5, IL-9, IL-10 and IL-13.

### Fusion Protein and Therapeutic Use Thereof

In another aspect, the present application also provides a fusion protein, which comprises a gp96 protein or variant thereof, and an additional peptide connected to the gp96 protein or variant thereof;
wherein, the variant has a sequence identity of at least 90%, such as at least 95%, at least 96%, at least 97%, at least 98%, at least 99%; or, has a substitution (preferably conservative substitution), addition or deletion of one or several (e.g., 1, 2, 3, 4, 5, 6, 7, 8 or 9) amino acids as compared with the gp96 protein, and retains the function of the gp96 protein;
the additional peptide is connected to the N-terminal and/or C-terminal of the gp96 protein or variant thereof, optionally via a linker (e.g., a peptide linker); and the additional peptide has a structure as shown in (GGGGS)ₙ₁C(GGGGS)ₙ₂, wherein the n1 and n2 are each independently selected from: 0, 1, 2, 3, 4, 5, 6, 7, 8, 9, 10. In some embodiments, the n1 and n2 are not 0 at the same time.

In some embodiments, the additional peptide has an amino acid sequence as set forth in SEQ ID NO: 6.

In some embodiments, the gp96 protein comprises or consists of an amino acid sequence as set forth in SEQ ID NO: 1 or 2. The sequence shown here does not comprise a methionine encoded by a start codon at its N-terminal. It is understood by those skilled in the art that the gp96 protein may also comprise or consist of the above amino acid sequence comprising a methionine encoded by a start codon at its N-terminal.

In some embodiments, the fusion protein comprises or consists of an amino acid sequence as set forth in SEQ ID NO: 4. The sequence shown here does not comprise a methionine encoded by a start codon at its N-terminal. It is understood by those skilled in the art that the gp96 protein may also comprise or consist of the above amino acid sequence that comprises a methionine encoded by a start codon at its N-terminal.

In some embodiments, the fusion protein may also comprise an additional protein tag, a targeting moiety, or any combination thereof.

Herein, the protein tag is well known in the art, examples of which include but are not limited to His, Flag, GST, MBP, HA, Myc, GFP or biotin, and those skilled in the art know how to select a suitable protein tag according to the desired purpose (e.g., purification, detection or tracing).

Herein, the term “targeting moiety” refers to a domain that can guide the fusion protein of the present invention to the desired location. The desired location can be a specific tissue, a specific cell, or even a specific intracellular location (e.g., a nucleus, a ribosome, an endoplasmic reticulum, a lysosome, or a peroxisome). Those skilled in the art know how to design a corresponding targeting moiety based on the characteristics of the desired location. In certain embodiments, the targeting moiety comprises a ligand, a receptor, or an antibody or binding domain thereof.

The fusion protein of the present invention is not limited by its production mode, for example, it can be produced by genetic engineering method (recombinant technology) or by chemical synthesis method.

In another aspect, the present application also provides an isolated nucleic acid molecule, which encodes the fusion protein as described above.

In another aspect, the present application also provides a vector, which comprises the isolated nucleic acid molecule as described above. In certain embodiments, the vector is a cloning vector or an expression vector (e.g., an insect cell expression vector). In certain embodiments, the vector of the present invention is, for example, a plasmid, a cosmid, a phage, a cosmid, and the like.

In another aspect, the present application also provides a host cell, which comprises the isolated nucleic acid molecule or vector as described above. Such host cells include, but are not limited to, prokaryotic cells such as Escherichia coli cells, and eukaryotic cells such as yeast cells, insect cells (e.g., Sf9 cells), plant cells and animal cells (e.g., mammalian cells, such as mouse cells, human cells, etc.).

It is easy to understand that the isolated nucleic acid molecule or vector as described above contained in the host cell comprises a nucleotide sequence encoding the fusion protein.

In some embodiments, the nucleotide sequence encoding the fusion protein is introduced into the host cell through a recombinant insect virus expression vector.

In some embodiments, the nucleotide sequence encoding the fusion protein is introduced into the host cell through a recombinant insect virus. In some embodiments, the recombinant insect virus is obtained by expressing or propagating it through insect cells using a recombinant insect virus expression vector.

In another aspect, the present application also provides a method for preparing the fusion protein as described above, which comprises culturing the host cell as described above under a condition that allows protein expression, and recovering the fusion protein from a culture of the cultured host cell.

In another aspect, the present application also provides a pharmaceutical composition, which comprises the fusion protein, isolated nucleic acid molecule, vector or host cell as described above, and a pharmaceutically acceptable carrier and/or excipient.

The pharmaceutical composition of the present invention can be formulated into any dosage form known in the medical field, for example, tablet, pill, suspension, emulsion, solution, gel, capsule, powder, granule, elixir, lozenge, suppository, injection (including injection solution, lyophilized powder), etc. In some embodiments, the pharmaceutical composition of the present invention can be formulated into injection solution or lyophilized powder.

In addition, the fusion protein, isolated nucleic acid molecule, vector or host cell of the present invention can be present in the pharmaceutical composition in a unit dose form for easy administration.

The pharmaceutical composition of the present invention can be administered by any suitable method known in the art, including but not limited to oral, buccal, sublingual, ocular, local, parenteral, rectal, intrathecal, intra-cisternal, inguinal, intravesical, topical (e.g., powder, ointment or drops), or nasal route. However, for many therapeutic uses, the preferred route/mode of administration is parenteral administration (e.g., intravenous injection, subcutaneous injection, intraperitoneal injection, intramuscular injection). The skilled person will understand that the route and/or mode of administration will vary depending on the intended purpose. In a preferred embodiment, the pharmaceutical composition of the present invention is administered by intravenous infusion or injection.

The pharmaceutical composition provided by the present invention can be administered alone or in combination, or in combination with an additional pharmaceutically active agent. This additional pharmaceutically active agent can be administered before, simultaneously with, or after the administration of the pharmaceutical composition of the present invention.

In certain embodiments, the pharmaceutical composition optionally further comprises an additional pharmaceutically active agent.

In certain embodiments, the additional pharmaceutically active agent is a drug for treating amyotrophic lateral sclerosis.

In another aspect, the present application also provides a use of the fusion protein, isolated nucleic acid molecule, vector, host cell or pharmaceutical composition as described above in the manufacture of a medicament for preventing and/or treating amyotrophic lateral sclerosis in a subject.

In certain embodiments, the pharmaceutical composition is used for one or more of the following:
(1) inducing a regulatory T cell;
(2) inhibiting the generation of a Th17 cell;
(3) inducing an increase in the number of Th2 cell;
(4) inhibiting the generation of a Th1 cell;
(5) reducing reactive oxygen species and oxidative stress in a motor neuron;
(6) reducing the expression of SOD1;
(7) restoring the function of dysfunctional mitochondria in a motor neuron;
(8) reducing denatured protein in a cell;
(9) reducing creatine kinase level and/or inhibiting creatine kinase activity, and upregulating creatine level;
(10) promoting the production of nerve growth factor;
(11) promoting the growth of a diseased motor nerve axon;
(12) improving axonal transport capacity.

In certain embodiments, the pharmaceutical composition is used for one or more of the following:
(1) inducing a regulatory T cell in a subject;
(2) inhibiting the generation of a Th17 cell in a subject;
(3) inducing an increase in the number of Th2 cell in a subject;
(4) inhibiting the generation of a Th1 cell in a subject;
(5) reducing reactive oxygen species and oxidative stress in a motor neuron in a subject;
(6) reducing the expression of SOD1 in a subject;
(7) restoring the function of dysfunctional mitochondria in a motor neuron in a subject;
(8) reducing denatured protein in a cell in a subject;
(9) reducing creatine kinase level and/or inhibiting creatine kinase activity, and upregulating creatine level in a subject;
(10) promoting the production of nerve growth factor in a subject;
(11) promoting the growth of a diseased motor nerve axon in a subject;
(12) improving axonal transport capacity in a subject.

In certain embodiments, the subject is a human or a mouse. In certain preferred embodiments, the subject is a human.

In certain embodiments, the regulatory T cell is a CD4+CD25+FOXP3+ regulatory T cell. In certain embodiments, the Th17 is a CD4+ T cell capable of producing IL-17 (interleukin 17). In certain embodiments, the Th1 is a CD4+ T cell capable of producing IFN-γ (interferon-γ), TNFβ (tumor necrosis factor β), granulocyte-macrophage colony-stimulating factor (GM-CSF), IL-2, lymphotoxin (LT). In certain embodiments, the Th2 is a CD4+ T cell capable of producing IL4, IL5, IL-9, IL-10 and IL-13.

In another aspect, the present application provides a method for preventing and/or treating amyotrophic lateral sclerosis, comprising: administering an effective amount of the fusion protein, isolated nucleic acid molecule, vector, host cell or pharmaceutical composition as described above to a subject in need thereof.

In certain embodiments, the method is used for one or more of the following:
(1) inducing a regulatory T cell;
(2) inhibiting the generation of a Th17 cell;
(3) inducing an increase in the number of Th2 cell;
(4) inhibiting the generation of a Th1 cell;
(5) reducing reactive oxygen species and oxidative stress in a motor neuron;
(6) reducing the expression of SOD1;
(7) restoring the function of dysfunctional mitochondria in a motor neuron;
(8) reducing denatured protein in a cell;
(9) reducing creatine kinase level and/or inhibiting creatine kinase activity, and upregulating creatine level;
(10) promoting the production of nerve growth factor;
(11) promoting the growth of a diseased motor nerve axon;
(12) improving axonal transport capacity.

In certain embodiments, the method is used for one or more of the following:
(1) inducing a regulatory T cell in a subject;
(2) inhibiting the generation of a Th17 cell in a subject;
(3) inducing an increase in the number of Th2 cell in a subject;
(4) inhibiting the generation of a Th1 cell in a subject;
(5) reducing reactive oxygen species and oxidative stress in a motor neuron in a subject;
(6) reducing the expression of SOD1 in a subject;
(7) restoring the function of dysfunctional mitochondria in a motor neuron in a subject;
(8) reducing denatured protein in a cell in a subject;
(9) reducing creatine kinase level and/or inhibiting creatine kinase activity, and upregulating creatine level in a subject;
(10) promoting the production of nerve growth factor in a subject;
(11) promoting the growth of a diseased motor nerve axon in a subject;
(12) improving axonal transport capacity in a subject.

In certain embodiments, the method further comprises administering an additional pharmaceutically active agent to the subject. This additional pharmaceutically active agent can be administered before, simultaneously with, or after the administration of the fusion protein, isolated nucleic acid molecule, vector, host cell, or pharmaceutical composition of the present invention.

In certain embodiments, the additional pharmaceutically active agent is a drug having a therapeutic effect on amyotrophic lateral sclerosis.

In certain embodiments, the subject is a human or a mouse. In certain preferred embodiments, the subject is a human.

In certain embodiments, the regulatory T cell is a CD4+CD25+FOXP3+ regulatory T cell. In certain embodiments, the Th17 is a CD4+ T cell capable of producing IL-17 (interleukin 17). In certain embodiments, the Th1 is a CD4+ T cell capable of producing IFN-γ (interferon-gamma), TNFβ (tumor necrosis factor β), granulocyte-macrophage colony-stimulating factor (GM-CSF), IL-2, lymphotoxin (LT). In certain embodiments, the Th2 is a CD4+ T cell capable of producing IL4, IL5, IL-9, IL-10, and IL-13.

### Definition of Terms

In the present application, unless otherwise specified, the scientific and technical terms used herein have the meanings commonly understood by those skilled in the art. In addition, the virology, biochemistry, and immunology laboratory operation steps used herein are all routine steps widely used in the corresponding fields. At the same time, in order to better understand the present invention, the definitions and explanations of relevant terms are provided below.

When the terms “for example”, “e.g.”, “such as”, “including”, “comprising” or variations thereof are used herein, these terms will not be considered as restrictive terms, but will be interpreted as meaning “but not limited to” or “not limited to”.

Unless otherwise specified herein or clearly contradicted by the context, the terms “a” and “an” and “the” and similar referents should be interpreted as covering the singular and plural in the context of describing the present invention (especially in the context of the following claims).

As used herein, the term “gp96”, also known as Grp94, is a member of the heat shock protein 90 family located on the endoplasmic reticulum membrane of cells. The gp96 protein consists of an N-terminal domain (N-terminal ATP binding domain), an M domain (charged middle domain), and a C-terminal domain (C-terminal homodimerization domain). gp96 is well known to those skilled in the art, and its sequence can be found in various public databases, such as the NCBI GENBANK database with the accession number: AAH66656.1.

As used herein, when referring to the amino acid sequence of the gp96 protein, it is described using the sequence as set forth in SEQ ID NO: 1. However, those skilled in the art understand that a mutation or variation can be naturally or artificially introduced into the amino acid sequence of gp96 without affecting its biological function. Therefore, in the present application, the term “gp96” and similar expressions shall include all such sequences, including, for example, the sequence as set forth in SEQ ID NO: 1 and its natural or artificial variants. Moreover, when describing a sequence fragment of the gp96 protein, it includes not only a sequence fragment of SEQ ID NO: 1, but also a corresponding sequence fragment in its natural or artificial variant.

As used herein, the term “isolated” or “being isolated” refers to “obtained” from a natural state by artificial means. If a certain “isolated” substance or component appears in nature, it may be that the natural environment in which it is located has changed, or the substance has been separated from the natural environment, or both. For example, a certain unisolated polynucleotide or polypeptide naturally exists in a living animal, and the same polynucleotide or polypeptide with high purity separated from this natural state is called isolated. The term “isolated” or “being isolated” does not exclude the presence of artificial or synthetic substances, nor does it exclude the presence of other impure substances that do not affect the activity of the substance.

As used herein, the term “vector” refers to a nucleic acid vehicle into which a polynucleotide can be inserted. When a vector can express the protein encoded by the inserted polynucleotide, the vector is called an expression vector. The vector can be introduced into a host cell by transformation, transduction or transfection, so that the genetic material elements it carries are expressed in the host cell. The vector is well known to those skilled in the art, including but not limited to: plasmid; phagemid; cosmid; artificial chromosome, such as yeast artificial chromosome (YAC), bacterial artificial chromosome (BAC) or P1-derived artificial chromosome (PAC); bacteriophage such as λ phage or M13 phage, and animal virus, etc. Animal virus that can be used as vector includes but is not limited to retrovirus (including lentivirus), adenovirus, adeno-associated virus, herpes virus (e.g., herpes simplex virus), poxvirus, baculovirus, papillomavirus, papovavirus (e.g., SV40). A vector can comprise a variety of elements that control expression, including but not limited to promoter sequence, transcription start sequence, enhancer sequence, selection element and reporter gene. In addition, the vector may also comprise a replication origin.

As used herein, the term “host cell” refers to a cell that can be used to introduce a vector, including but not limited to prokaryotic cell such as Escherichia coli or Bacillus subtilis, fungal cell such as yeast cell or Aspergillus, insect cell such as S2 Drosophila cell or Sf9, or animal cell such as fibroblast, CHO cell, COS cell, NSO cell, HeLa cell, BHK cell, HEK 293 cell or human cell.

As used herein, the term “identity” is used to refer to the matching of sequences between two polypeptides or between two nucleic acids. When a certain position in the two compared sequences is occupied by the same nucleotide or amino acid residue (e.g., a certain position in each of the two DNA molecules is occupied by adenine nucleotide, or a certain position in each of the two polypeptides is occupied by lysine), then the molecules are identical at that position. The “percentage identity” between two sequences is a function of the number of matching positions shared by the two sequences divided by the number of positions compared×100. For example, if 6 out of 10 positions in two sequences match, then the two sequences have an identity of 60%. For example, the DNA sequences CTGACT and CAGGTT have an identity of 50% (3 out of a total of 6 positions match). Typically, the comparison is made when two sequences are aligned to produce maximum identity. Such alignment can be made by the method of Needleman et al., J Mol Biol. 48:444-453 (1970) conveniently using, for example, a computer program such as the Align program (DNAs, Inc.). The percentage identity between two amino acid sequences can also be determined using the algorithm of E. Meyers and W. Miller (Comput. Appl Biosci., 4:11-17 (1988)), which has been incorporated into the ALIGN program (version 2.0), using a PAM120 weight residue table, a gap length penalty of 12, and a gap penalty of 4. In addition, the percentage identity between two amino acid sequences can be determined using the algorithm of Needleman and Wunsch (J Mol Biol. 48:444-453 (1970)), which has been integrated into the GAP program of the GCG software package (available at www.gcg.com), using Blossum 62 matrix or PAM250 matrix, a gap weight of 16, 14, 12, 10, 8, 6 or 4 and a length weight of 1, 2, 3, 4, 5 or 6.

As used herein, the term “conservative substitution” refers to an amino acid substitution that does not adversely affect or change the expected properties of the protein/polypeptide comprising the amino acid sequence. For example, the conservative substitution can be introduced by standard techniques known in the art, such as site-directed mutagenesis and PCR-mediated mutagenesis. Conservative amino acid substitutions include substitutions in which amino acid residues are replaced with amino acid residues having similar side chains, such as substitutions performed with residues physically or functionally similar to the corresponding amino acid residues (e.g., having similar size, shape, charge, chemical properties, including the ability to form covalent bond or hydrogen bond, etc.). Families of amino acid residues with similar side chains have been defined in the art. These families include amino acids with basic side chains (e.g., lysine, arginine, and histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), β-branched side chains (e.g., threonine, valine, isoleucine), and aromatic side chains (e.g., tyrosine, phenylalanine, tryptophan, histidine). Therefore, it is preferred to replace the corresponding amino acid residue with another amino acid residue from the same side chain family. Methods for identifying conservative substitutions of amino acids are well known in the art (see, for example, Brummell et al., Biochem. 32:1180-1187 (1993); Kobayashi et al. Protein Eng. 12 (10): 879-884 (1999); and Burks et al. Proc. Natl Acad. Set USA 94:412-417 (1997), which are incorporated herein by reference).

The twenty conventional amino acids referred to herein are written in accordance with conventional usage. See, for example, Immunology-A Synthesis (2nd Edition, E. S. Golub and D. R. Gren, Eds., Sinauer Associates, Sunderland, Mass. (1991)), which is incorporated herein by reference. In the present application, the terms “polypeptide” and “protein” have the same meaning and are used interchangeably. And in the present application, amino acids are generally represented by single-letter and three-letter abbreviations known in the art. For example, alanine can be represented by A or Ala.

As used herein, the term “subject” includes, but is not limited to, various animals, particularly mammals, such as humans or mouse. In certain embodiments, the subject (e.g., human or mouse) suffers from amyotrophic lateral sclerosis.

As used herein, the term “pharmaceutically acceptable carrier and/or excipient” refers to a carrier and/or excipient that is pharmacologically and/or physiologically compatible with the subject and the active ingredient, which is well known in the art (see, for example, Remington's Pharmaceutical Sciences. Edited by Gennaro AR, 19th ed. Pennsylvania: Mack Publishing Company, 1995), and includes, but is not limited to, pH regulator, surfactant, ionic strength enhancer, agent for maintaining osmotic pressure, agent for delaying absorption, diluent, adjuvant, preservative, stabilizer, and the like. For example, the pH regulator includes, but is not limited to, phosphate buffer. The surfactant includes, but is not limited to, cationic, anionic or nonionic surfactant, such as Tween-80. The ionic strength enhancer includes, but is not limited to, sodium chloride. The agent for maintaining osmotic pressure includes, but is not limited to, sugar, NaCl, and the like. The agent for delaying absorption includes, but is not limited to, monostearate and gelatin. The diluent includes, but is not limited to, water, aqueous buffer (e.g., buffered saline), alcohol and polyol (e.g., glycerol), etc. The adjuvant includes, but is not limited to, aluminum adjuvant (e.g., aluminum hydroxide), Freund's adjuvant (e.g., complete Freund's adjuvant), etc. The preservative includes, but is not limited to, various antibacterial and antifungal agent, such as thimerosal, 2-phenoxyethanol, paraben, chlorobutanol, phenol, sorbic acid, etc. The stabilizer has the meaning commonly understood by those skilled in the art, which can stabilize the desired activity of the active ingredient in the drug (e.g., the inhibitory activity on PSD-95 ubiquitination), including but not limited to, sodium glutamate, gelatin, SPGA, sugar (e.g., sorbitol, mannitol, starch, sucrose, lactose, dextran, or glucose), amino acid (e.g., glutamic acid, glycine), protein (e.g., dried whey, albumin or casein) or degradation product thereof (e.g., lactalbumin hydrolysate), etc.

As used herein, the term “treatment” refers to treating or curing a disease (e.g., amyotrophic lateral sclerosis), delaying the onset of one or more symptoms of the disease, and/or delaying the progression of the disease.

As used herein, the term “effective amount” refers to an amount that is effective in achieving the intended purpose. For example, a therapeutically effective amount may be an amount that is effective or sufficient to treat or cure a disease (e.g., amyotrophic lateral sclerosis), delay the onset of one or more symptoms of the disease, and/or delay the progression of the disease. Such an effective amount may be readily determined by a person skilled in the art or a physician and may be related to the intended purpose, the general health conditions, age, gender, weight of the subject, severity of the disease to be treated, complications, mode of administration, etc. The determination of such an effective amount is well within the capabilities of a person skilled in the art.

### Beneficial Effects of the Invention

The gp96 protein or the fusion protein constructed therefrom of the present invention has the effects of reducing active oxygen species and oxidative stress in motor nerve cells, reducing the content of denatured proteins in cells, inducing the regulatory T cells, reducing the number of Th17 cells, downregulating Th1 and upregulating Th2 immunity, inhibiting neuroinflammation, restoring the function of dysfunctional mitochondria in motor nerve cells, inhibiting creatine kinase activity and upregulating creatine level, promoting the production of nerve growth factor, promoting the growth of diseased motor nerve axons, and/or improving the transport capacity of axons, and can be effectively used for the treatment of amyotrophic lateral sclerosis, and has important application value in treating amyotrophic lateral sclerosis or alleviating the symptoms of amyotrophic lateral sclerosis.

The embodiments of the present invention will be described in detail below with reference to the drawings and examples, but those skilled in the art will understand that the following drawings and examples are only used to illustrate the present invention, not to limit the scope of the present invention. According to the following detailed description of the drawings and preferred embodiments, the various objects and advantages of the present invention will become apparent to those skilled in the art.

### BRIEF DESCRIPTION OF THE DRAWINGS

FIG. 1 shows the numbers of regulatory T cells, Th17, Th1 and Th2 cells in the peripheral blood of mice immunized with gp96 protein, as detected by flow cytometry.

FIG. 2 shows the results of ELISA detection of the contents of reactive oxygen species (ROS), SOD1 and creatine kinase in mouse serum.

FIG. 3 shows the change of mitochondrial membrane potential detected by flow cytometry.

FIG. 4 shows the immunofluorescence detection results of the content of nerve growth factor (indicated by the average fluorescence intensity), the number of motor neurons, the length of nerve cell axons, the number of astrocytes and the number of microglia in the spinal cord of mice.

FIG. 5 shows the neurological function score of mice.

FIG. 6 shows the motor function of mice evaluated by the rotating rod test.

FIG. 7 shows the motor function of mice evaluated by the hanging wire test.

FIG. 8 shows the grip strength of hind feet of mice evaluated by the grip strength tester.

FIG. 9 shows the body weight and survival rate of mice.

### SEQUENCE INFORMATION

The description of the sequences involved in the present application is provided in the following table.

TABLE 1
Sequence information

| SEQ ID NO: | Description of sequence |
|---|---|
| 1 | pgp96 amino acid sequence |
| | DDEVDVDGTVEEDLGKSREGSRTDDEVVQREEEAIQLDGLNASQIRELRE |
| | KSEKFAFQAEVNRMMKLIINSLYKNKEIFLRELISNASDALDKIRLISLTDEN |
| | ALSGNEELTVKIKCDKEKNLLHVTDTGVGMTREELVKNLGTIAKSGTSEFL |
| | NKMTEAQEDGQSTSELIGQFGVGFYSAFLVADKVIVTSKHNNDTQHIWES |
| | DSNEFSVIADPRGNTLGRGTTITLVLKEEASDYLELDTIKNLVKKYSQFINF |
| | PIYVWSSKTETVEEPMEEEEAAKEEKEESDDEAAVEEEEEEKKPKTKKVEK |
| | TVWDWELMNDIKPIWQRPSKEVEEDEYKAFYKSFSKESDDPMAYIHFTAE |
| | GEVTFKSILFVPTSAPRGLFDEYGSKKSDYIKLYVRRVFITDDFHDMMPKY |
| | LNFVKGVVDSDDLPLNVSRETLQQHKLLKVIRKKLVRKTLDMIKKIADDK |
| | YNDTFWKEFGTNIKLGVIEDHSNRTRLAKLLRFQSSHHPTDITSLDQYVER |
| | MKEKQDKIYFMAGSSRKEAESSPFVERLLKKGYEVIYLTEPVDEYCIQALP |
| | EFDGKRFQNVAKEGVKFDESEKTKESREAVEKEFEPLLNWMKDKALKDKI |
| | EKAVVSQRLTESPCALVASQYGWSGNMERIMKAQAYQTGKDISTNYYAS |
| | QKKTFEINPRHPLIRDMLRRIKEDEDDKTVLDLAVVLFETATLRSGYLLPDT |
| | KAYGDRIERMLRLSLNIDPDAKVEEEPEEEPEETAEDTTEDTEQDEDEEMD |
| | VGTDEEEETAKESTAEKDEL |
| 2 | rgp96 amino acid sequence |
| | DDEVDVDGTVEEDLGKSREGSRTDDEVVQREEEAIQLDGLNASQIRELRE |
| | KSEKFAFQAEVNRMMKLIINSLYKNKEIFLRELISNASDALDKIRLISLTDEN |
| | ALSGNEELTVKIKCDKEKNLLHVTDTGVGMTREELVKNLGTIAKSGTSEFL |
| | NKMTEAQEDGQSTSELIGQFGVGFYSAFLVADKVIVTSKHNNDTQHIWES |
| | DSNEFSVIADPRGNTLGRGTTITLVLKEEASDYLELDTIKNLVKKYSQFINF |
| | PIYVWSSKTETVEEPMEEEEAAKEEKEESDDEAAVEEEEEEKKPKTKKVEK |
| | TVWDWELMNDIKPIWQRPSKEVEEDEYKAFYKSFSKESDDPMAYIHFTAE |
| | GEVTFKSILFVPTSAPRGLFDEYGSKKSDYIKLYVRRVFITDDFHDMMPKY |
| | LNFVKGVVDSDDLPLNVSRETLQQHKLLKVIRKKLVRKTLDMIKKIADDK |
| | YNDTFWKEFGTNIKLGVIEDHSNRTRLAKLLRFQSSHHPTDITSLDQYVER |
| | MKEKQDKIYFMAGSSRKEAESSPFVERLLKKGYEVIYLTEPVDEYCIQALP |
| | EFDGKRFQNVAKEGVKFDESEKTKESREAVEKEFEPLLNWMKDKALKDKI |
| | EKAVVSQRLTESPCALVASQYGWSGNMERIMKAQAYQTGKDISTNYYAS |
| | QKKTFEINPRHPLIRDMLRRIKEDEDDKTVLDLAVVLFETATLRSGYLLPDT |
| | KAYGDRIERMLRLSLNIDPDAKVEEEPEEEPEETAEDTTEDTEQDEDEEMD |
| | VGTDEEEETAKESTAE |
| 3 | nucleotide sequence encoding rpg96 |
| | GACGATGAAGTTGATGTGGATGGTACAGTAGAAGAGGATCTGGGTAAA |
| | AGTAGAGAAGGATCAAGGACGGATGATGAAGTAGTACAGAGAGAGGA |
| | AGAAGCTATTCAGTTGGATGGATTAAATGCATCACAAATAAGAGAACT |
| | TAGAGAGAAGTCGGAAAAGTTTGCCTTCCAAGCCGAAGTTAACAGAAT |
| | GATGAAACTTATCATCAATTCATTGTATAAAAATAAAGAGATTTTCCTG |
| | AGAGAACTGATTTCAAATGCTTCTGATGCTTTAGATAAGATAAGGCTAA |
| | TATCACTGACTGATGAAAATGCTCTTTCTGGAAATGAGGAACTAACAGT |
| | CAAAATTAAGTGTGATAAGGAGAAGAACCTGCTGCATGTCACAGACAC |
| | CGGTGTAGGAATGACCAGAGAAGAGTTGGTTAAAAACCTTGGTACCAT |
| | AGCCAAATCTGGGACAAGCGAGTTTTTAAACAAAATGACTGAAGCACA |
| | GGAAGATGGCCAGTCGTCTTCTGAATTGATTGGCCAGTTTGGTGTCGGT |
| | TTCTATTCCGCCTTCCTTGTAGCAGATAAGGTTATTGTCACTTCAAAACA |
| | CAACAACGATACCCAGCACATCTGGGAGTCTGACTCCAATGAATTTTCT |
| | GTAATTGCTGACCCAAGAGGAAACACTCTAGGACGGGGAACGACAATT |
| | ACCCTTGTCTTAAAAGAAGAAGCATCTGATTACCTTGAATTGGATACAA |
| | TTAAAAATCTCGTCAAAAAATATTCACAGTTCATAAACTTTCCTATTTAT |
| | GTATGGAGCAGCAAGACTGAAACTGTTGAGGAGCCCATGGAGGAAGAA |
| | GAAGCAGCCAAAGAAGAGAAAGAAGAATCTGATGATGAAGCTGCAGT |
| | AGAGGAAGAAGAAGAAGAAAAGAAACCAAAGACTAAAAAAGTTGAAA |
| | AAACTGTCTGGGACTGGGAACTTATGAATGATATCAAACCAATATGGC |
| | AGAGACCATCAAAAGAAGTAGAAGAAGATGAATACAAAGCTTTCTACA |
| | AATCATTTTCAAAGGAAAGTGATGACCCCATGGCTTATATTCACTTTAC |
| | TGCTGAAGGGGAAGTTACCTTCAAATCAATTTTATTTGTACCCACATCT |
| | GCTCCACGTGGTCTGTTTGACGAATATGGATCTAAAAAGAGCGATTACA |
| | TTAAGCTCTATGTGCGCCGTGTATTCATCCCAGACGACTTCCATGATAT |
| | GATGCCTAAATACCTCAATTTTGTCAAGGGTGTGGTGGACTCAGATGAT |
| | CTCCCCTTGAATGTTTCCCGCGAGACTCTTCAGCAACATAAACTGCTTA |
| | AGGTGATTAGGAAGAAGCTTGTTCGTAAAACGCTGGACATGATCAAGA |
| | AGATTGCTGATGATAAATACAATGATACTTTTTGGAAAGAATTTGGTAC |
| | CAACATCAAGCTTGGTGTGATTGAAGACCACTCGAATCGAACACGTCTT |
| | GCTAAACTTCTTAGGTTCCAGTCTTCTCATCATCCAACTGACATTACTAG |
| | CCTAGACCAGTATGTGGAAAGAATGAAGGAAAAACAAGACAAAATCTA |
| | CTTCATGGCTGGGTCCAGCAGAAAAGAGGCTGAATCTTCTCCATTTGTT |
| | GAGCGACTTCTGAAAAAGGGCTATGAAGTTATTTACCTCACAGAACCTG |
| | TGGATGAATACTGTATTCAGGCCCTTCCCGAATTTGATGGGAAGAGGTT |
| | CCAGAATGTTGCCAAGGAAGGAGTGAAGTTCGATGAAAGTGAGAAAAC |
| | TAAGGAGAGTCGTGAAGCAGTTGAGAAAGAATTTGAGCCTCTGCTGAA |
| | TTGGATGAAAGATAAAGCCCTTAAGGACAAGATTGAAAAGGCTGTGGT |
| | GTCTCAGCGCCTGACAGAATCTCCGTGTGCTTTGGTGGCCAGCCAGTAC |
| | GGATGGTCTGGCAACATGGAGAGAATCATGAAAGCACAAGCGTACCAA |
| | ACGGGCAAGGACATCTCTACAAATTACTATGCGAGTCAGAAGAAAACA |
| | TTTGAAATTAATCCCAGACACCCGCTGATCAGAGACATGCTTCGACGAA |
| | TTAAGGAAGATGAAGATGATAAAACAGTTTTGGATCTTGCTGTGGTTTT |
| | GTTTGAAACAGCAACGCTTCGGTCAGGGTATCTTTTACCAGACACTAAA |
| | GCATATGGAGATAGAATAGAAAGAATGCTTCGCCTCAGTTTGAACATT |
| | GACCCTGATGCAAAGGTGGAAGAAGAGCCCGAAGAAGAACCTGAGGA |
| | GACAGCAGAAGACACAACAGAAGACACAGAGCAAGACGAAGATGAAG |
| | AAATGGATGTGGGAACAGATGAAGAAGAAGAAACAGCAAAGGAATCT |
| | ACAGCTGAA |
| 4 | gp96-plus amino acid sequence |
| | GGGGSCGGGGSMDDEVDVDGTVEEDLGKSREGSRTDDEVVQREEEAIQL |
| | DGLNASQIRELREKSEKFAFQAEVNRMMKLIINSLYKNKEIFLRELISNASD |
| | ALDKIRLISLTDENALSGNEELTVKIKCDKEKNLLHVTDTGVGMTREELVK |
| | NLGTIAKSGTSEFLNKMTEAQEDGQSTSELIGQFGVGFYSAFLVADKVIVTS |
| | KHNNDTQHIWESDSNEFSVIADPRGNTLGRGTTITLVLKEEASDYLELDTIK |
| | NLVKKYSQFINFPIYVWSSKTETVEEPMEEEEAAKEEKEESDDEAAVEEEE |
| | EEKKPKTKKVEKTVWDWELMNDIKPIWQRPSKEVEEDEYKAFYKSFSKES |
| | DDPMAYIHFTAEGEVTFKSILFVPTSAPRGLFDEYGSKKSDYIKLYVRRVFI |
| | TDDFHDMMPKYLNFVKGVVDSDDLPLNVSRETLQQHKLLKVIRKKLVRK |
| | TLDMIKKIADDKYNDTFWKEFGTNIKLGVIEDHSNRTRLAKLLRFQSSHHP |
| | TDITSLDQYVERMKEKQDKIYFMAGSSRKEAESSPFVERLLKKGYEVIYLT |
| | EPVDEYCIQALPEFDGKRFQNVAKEGVKFDESEKTKESREAVEKEFEPLLN |
| | WMKDKALKDKIEKAVVSQRLTESPCALVASQYGWSGNMERIMKAQAYQ |
| | TGKDISTNYYASQKKTFEINPRHPLIRDMLRRIKEDEDDKTVLDLAVVLFET |
| | ATLRSGYLLPDTKAYGDRIERMLRLSLNIDPDAKVEEEPEEEPEETAEDTTE |
| | DTEQDEDEEMDVGTDEEEETAKESTAE |
| 5 | nucleotide sequence encoding gp96-plus |
| | GGTGGTGGTGGTTCCTGCGGTGGTGGTGGTTCCATGGACGATGAAGTTG |
| | ATGTGGATGGTACAGTAGAAGAGGATCTGGGTAAAAGTAGAGAAGGAT |
| | CAAGGACGGATGATGAAGTAGTACAGAGAGAGGAAGAAGCTATTCAGT |
| | TGGATGGATTAAATGCATCACAAATAAGAGAACTTAGAGAGAAGTCGG |
| | AAAAGTTTGCCTTCCAAGCCGAAGTTAACAGAATGATGAAACTTATCAT |
| | CAATTCATTGTATAAAAATAAAGAGATTTTCCTGAGAGAACTGATTTCA |
| | AATGCTTCTGATGCTTTAGATAAGATAAGGCTAATATCACTGACTGATG |
| | AAAATGCTCTTTCTGGAAATGAGGAACTAACAGTCAAAATTAAGTGTG |
| | ATAAGGAGAAGAACCTGCTGCATGTCACAGACACCGGTGTAGGAATGA |
| | CCAGAGAAGAGTTGGTTAAAAACCTTGGTACCATAGCCAAATCTGGGA |
| | CAAGCGAGTTTTTAAACAAAATGACTGAAGCACAGGAAGATGGCCAGT |
| | CGTCTTCTGAATTGATTGGCCAGTTTGGTGTCGGTTTCTATTCCGCCTTC |
| | CTTGTAGCAGATAAGGTTATTGTCACTTCAAAACACAACAACGATACCC |
| | AGCACATCTGGGAGTCTGACTCCAATGAATTTTCTGTAATTGCTGACCC |
| | AAGAGGAAACACTCTAGGACGGGGAACGACAATTACCCTTGTCTTAAA |
| | AGAAGAAGCATCTGATTACCTTGAATTGGATACAATTAAAAATCTCGTC |
| | AAAAAATATTCACAGTTCATAAACTTTCCTATTTATGTATGGAGCAGCA |
| | AGACTGAAACTGTTGAGGAGCCCATGGAGGAAGAAGAAGCAGCCAAA |
| | GAAGAGAAAGAAGAATCTGATGATGAAGCTGCAGTAGAGGAAGAAGA |
| | AGAAGAAAAGAAACCAAAGACTAAAAAAGTTGAAAAAACTGTCTGGG |
| | ACTGGGAACTTATGAATGATATCAAACCAATATGGCAGAGACCATCAA |
| | AAGAAGTAGAAGAAGATGAATACAAAGCTTTCTACAAATCATTTTCAA |
| | AGGAAAGTGATGACCCCATGGCTTATATTCACTTTACTGCTGAAGGGGA |
| | AGTTACCTTCAAATCAATTTTATTTGTACCCACATCTGCTCCACGTGGTC |
| | TGTTTGACGAATATGGATCTAAAAAGAGCGATTACATTAAGCTCTATGT |
| | GCGCCGTGTATTCATCCCAGACGACTTCCATGATATGATGCCTAAATAC |
| | CTCAATTTTGTCAAGGGTGTGGTGGACTCAGATGATCTCCCCTTGAATG |
| | TTTCCCGCGAGACTCTTCAGCAACATAAACTGCTTAAGGTGATTAGGAA |
| | GAAGCTTGTTCGTAAAACGCTGGACATGATCAAGAAGATTGCTGATGA |
| | TAAATACAATGATACTTTTTGGAAAGAATTTGGTACCAACATCAAGCTT |
| | GGTGTGATTGAAGACCACTCGAATCGAACACGTCTTGCTAAACTTCTTA |
| | GGTTCCAGTCTTCTCATCATCCAACTGACATTACTAGCCTAGACCAGTA |
| | TGTGGAAAGAATGAAGGAAAAACAAGACAAAATCTACTTCATGGCTGG |
| | GTCCAGCAGAAAAGAGGCTGAATCTTCTCCATTTGTTGAGCGACTTCTG |
| | AAAAAGGGCTATGAAGTTATTTACCTCACAGAACCTGTGGATGAATACT |
| | GTATTCAGGCCCTTCCCGAATTTGATGGGAAGAGGTTCCAGAATGTTGC |
| | CAAGGAAGGAGTGAAGTTCGATGAAAGTGAGAAAACTAAGGAGAGTC |
| | GTGAAGCAGTTGAGAAAGAATTTGAGCCTCTGCTGAATTGGATGAAAG |
| | ATAAAGCCCTTAAGGACAAGATTGAAAAGGCTGTGGTGTCTCAGCGCC |
| | TGACAGAATCTCCGTGTGCTTTGGTGGCCAGCCAGTACGGATGGTCTGG |
| | CAACATGGAGAGAATCATGAAAGCACAAGCGTACCAAACGGGCAAGG |
| | ACATCTCTACAAATTACTATGCGAGTCAGAAGAAAACATTTGAAATTAA |
| | TCCCAGACACCCGCTGATCAGAGACATGCTTCGACGAATTAAGGAAGA |
| | TGAAGATGATAAAACAGTTTTGGATCTTGCTGTGGTTTTGTTTGAAACA |
| | GCAACGCTTCGGTCAGGGTATCTTTTACCAGACACTAAAGCATATGGAG |
| | ATAGAATAGAAAGAATGCTTCGCCTCAGTTTGAACATTGACCCTGATGC |
| | AAAGGTGGAAGAAGAGCCCGAAGAAGAACCTGAGGAGACAGCAGAAG |
| | ACACAACAGAAGACACAGAGCAAGACGAAGATGAAGAAATGGATGTG |
| | GGAACAGATGAAGAAGAAGAAACAGCAAAGGAATCTACAGCTGAA |
| 6 | Linker amino acid sequence |
| | GGGGSCGGGGS |
| 7 | Primer F1 |
| | GGAATTCATGGACGATGAAGTTGAT |
| 8 | Primer R1 |
| | GCTCTAGACTATTAGAATTCATCTTTTTC |

### Specific Models for Carrying Out the Invention

The present invention is now described with reference to the following examples which are intended to illustrate the present invention (but not to limit the present invention) and are not intended to limit the scope of protection claimed by the present invention.

The experimental methods in the following examples were conventional methods unless otherwise specified.

The materials, reagents and so on used in the following examples could be obtained from commercial channels unless otherwise specified.

The quantitative experiments in the following examples were set up for three repeated experiments, and the results were averaged.

### Experimental Materials:

hSOD1-G93A transgenic mice were purchased from Jackson Laboratory, USA, with Cat. NO.: 004435. Sf9 cells were product of Invitrogen, with Cat. NO.: 11496-015. Plasmid pFastBac™ 1 was a product of Invitrogen, with Cat. NO.: 10359-016. DH10Bac™ competent cells were product of Invitrogen, with Cat. NO.: 10361-012. Insect-XPRESS™ Protein-free Insect Cells medium with L-Glutamine was product of LONZA, with Cat. NO.: 12-730Q. Ultrafiltration tubes were product of Merck Millipore, with Cat. NO.: UFC905096. ELISA kit was product of eBioscience, with Cat. NO.: BMS614INST. Ni affinity chromatography prepacked column was product of Aladdin, with Cat. NO.: N5289-01. Superdex 200 10/300 GL molecular sieve chromatography column was product of GE Company, with Cat. NO.: 17517501. Escherichia coli DH10Bac competent cells were product of Beijing Yuanpinghao Biotechnology Co., Ltd., with Cat. NO.: CL108-01.

### Example 1: Extraction of pgp96

The steps for extracting heat shock protein gp96 from tissues (hereinafter referred to as pgp96, which had an amino acid sequence as set forth in SEQ ID NO: 1 and comprised a methionine at the N-terminal) were as follows:
(1) Ex vivo human placental tissues were taken, cut it into pieces, added with Solution A (PMSF and NaHCO₃ were dissolved in water to obtain Solution A; in Solution A, the concentration of PMSF was 1 mM, and the concentration of NaHCO₃ was 30 mM) at a mass volume ratio of 1 g:4 mL, and then ground with a glass homogenizer.
(2) After step (1) was completed, centrifugation was performed at 16500 g for 1h to obtain Supernatant A.
(3) After step (2) was completed, Supernatant A was taken, centrifuged at 16500 g for 50 min to obtain Supernatant B.
(4) After step (3) was completed, Supernatant B was taken, added with Solution B (20 mM Tris-HCl (pH7.4) solution) at a volume ratio of 9:1, and mixed well to obtain a loading solution.
(5) After step (4) was completed, the loading solution was loaded onto a ConA agarose gel column.
(6) After step (5) was completed, the ConA agarose gel column was eluted with a cleaning solution, the ultraviolet absorption value was monitored in real time during the elution process, and the detection wavelength was 280 nm, until the ultraviolet absorption value of the eluted product was lower than 0.01.
(7) After step (6) was completed, the ConA agarose gel column was eluted with Solution C (20 mM Tris-HCl (pH 7.4) solution, the solutes and concentrations thereof were as follows: 10% (10 g/100 ml) α-D-pyranose glucose, 500 mM NaCl, 1 mM PMSF), the first 0.5 column volume of the solution that passed through the column was discarded, and then 1 column volume of the solution that passed through the column later was collected; after the ConA agarose gel column was incubated for 50 minutes, then 1.5 column volumes of the solution that passed through the column were collected. The two collected solutions were combined to obtain a ConA eluate.
(8) After step (7) was completed, the ConA eluate was loaded onto a Hitrap Q anion exchange column.
(9) After step (8) was completed, linear gradient elution was performed with 12 mM PBS buffer (pH 7.4, containing NaCl) at a flow rate of 1 mL/min. Gradient elution procedure was as follows: in 12 mM PBS buffer (pH 7.4), the NaCl content was increased from 300 mM to 800 mM at a uniform rate, and a linear gradient elution was performed for 20 column volumes. The eluate with a NaCl content of 1.400˜450 mM was collected and combined, which was Eluate A.
(10) After step (9) was completed, Eluate A was taken and ultrafiltered and concentrated using ultrafiltration Tube A to obtain a pgp96 solution. In the pgp96 solution, the pgp96 concentration was 5 mg/mL.

### Example 2: Preparation of Recombinant Heat Shock Protein gp96 (Abbreviated as rgp96)

I. Construction of Recombinant Plasmid pFastBac1-gp96

1. RNA of HepG2 cells was extracted using the Trizol-based method, and then it underwent reverse transcription to obtain cDNA.

2. According to the sequence of human gp96 gene (GenBank No. AY040226.1), primers F1: 5′-GGAATTCATGGACGATGAAGTTGAT-3′ (SEQ ID NO: 7, in which the restriction endonuclease EcoRI sequence recognition was underlined) and R1: 5′-GCTCTAGACTATTAGAATTCATCTTTTTC-3′ (SEQ ID NO: 8, in which the restriction endonuclease XbaI recognition sequence was underlined) were artificially synthesized.

3. After steps 1 and 2 were completed, PCR amplification was performed using the cDNA obtained in step 1 as a template and the F1 and R1 synthesized in step 2 as primers to obtain a PCR amplification product.

4. The PCR amplification product was double-digested with restriction endonucleases EcoRI and XbaI, and the digestion product was recovered.

5. The plasmid pFastBac™ 1 was digested with restriction endonucleases EcoRI and XbaI, and the vector backbone of about 4700 bp was recovered.

6. The digestion product was ligated to the vector backbone to obtain a ligation product.

7. The ligation product obtained in step 6 was transformed into Escherichia coli DH10Bac competent cells to obtain recombinant Escherichia coli, and then the plasmid of the recombinant Escherichia coli was extracted to obtain the recombinant plasmid pFastBac1-gp96, which comprised the coding sequence of rgp96 (which had the amino acid sequence as set forth in SEQ ID NO: 2 and comprised a methionine at the N-terminal).

According to the sequencing results, the structure of the recombinant plasmid pFastBac1-gp96 was described as follows: the fragment between the EcoRI and XbaI recognition sequences of the plasmid pFastBac1 (the plasmid pFastBac1 was cut into a large fragment and a small fragment by restriction endonucleases EcoRI and XbaI, and the fragment is the small fragment) was replaced with a double-stranded DNA molecule encoding rgp96 (which comprised the nucleotide sequence as set forth in SEQ ID NO: 3, and comprised ATG at the 5′ end and TAA at the 3′ end).

II. Expression of rgp96

1. Sf9 cells were transfected with the recombinant plasmid pFastBac1-gp96 as constructed in step I (approximately 4 μg of recombinant plasmid pFastBac1-gp96 per 1×10⁶ Sf9 cells). During the co-transfection process, the transfection reagent was Cellfectin II reagent, the culture medium was Insect-XPRESS Protein-Free™ Insect Cells medium with L-Glutamine, the incubation was performed at 27° C. for 72 hours, and the supernatant after configuration was P1 generation virus.

2. Sf9 cell Suspension 1 (containing 1×10⁸ Sf9 cells) was cultured at 27° C. for 8 to 10 hours to obtain cultured cells; then the P1 generation virus (with dosage of 0.05 to 0.1 MOI) was added to the cultured cells, incubated at 27° C. for 72 hours, and centrifuged at 4000 rpm for 5 minutes, and the supernatant was P2 generation virus.

3. The P2 generation virus (with dosage of 0.058 to 0.1 MOI) was added to Sf9 cell Suspension 2 (containing 1.6×10⁸ Sf9 cells), cultured at 27° C. and 100 to 120 rpm for 72 hours, and centrifuged at 4000 rpm for 5 minutes, and the supernatant was P3 generation virus.

III. Purification of rgp96

1. The P3 generation virus (with dosage of 5 MOI) was added to 300 ml of Sf9 cell Suspension 3 (containing 4.5×10⁸ Sf9 cells), cultured at 27° C. and 100 to 120 rpm for 72 hours to obtain a suspension.

2. The suspension was taken, and centrifuged at 7000 rpm for 20 minutes to obtain Supernatant 1.

3. The Supernatant 1 was taken, and filtered through a 0.22 mm filter membrane to obtain a sample solution.

4. The sample solution was loaded onto a HiTrap-Q Sepharose ion exchange chromatography column (flow rate of 1 mL/min), which was then eluted with 5 mL of pH7.5, 200 mM PBS buffer (flow rate of 1 mL/min); then eluted with 10 mL of pH7.5, 300 mM PBS buffer (flow rate of 1 mL/min); and finally eluted with 3 mL of pH7.5, 600 mM PBS buffer (flow rate of 1 mL/min), and the solution passed through the column was collected and underwent ultrafiltration concentration using an ultrafiltration tube with a molecular weight cutoff of 50 KD to obtain about 1 mL of a concentrated solution, which contained rgp96.

5. The concentrated solution obtained in step 4 was loaded onto a Superdex 200 10/300GL molecular sieve chromatography column (flow rate of 0.25 mL/min), which was then eluted with pH 7.5, 150 mM PBS buffer (flow rate of 0.25 mL/min), and the penetration solution at the 9^{th} to 12^{th} mL was collected and further underwent ultrafiltration concentration using an ultrafiltration tube with a molecular weight cutoff of 50 KD to obtain a rgp96 solution. The protein concentration of the rgp96 solution was determined by the BCA assay, and finally the solution was aliquoted and stored at −80° C.

### Example 3: Preparation of gp96-Plus Protein

### I. Construction of Recombinant Plasmid

The coding nucleotide sequence of heat shock protein gp96 and the coding nucleotide sequence of flexible linker (with amino acid sequence set forth in SEQ ID NO: 6) were ligated in series by artificial polynucleotide synthesis (the synthesis was entrusted to GenScript Biotech Co., Ltd.) to obtain a target nucleotide fragment (which had the nucleotide sequence as set forth in SEQ ID NO: 5, and comprised ATG at the 5′ end and TAA at the 3′ end) encoding gp96-plus protein (which had the amino acid sequence as set forth in SEQ ID NO: 4 and comprised a methionine at the N-terminal), and then the target fragment was ligated to the insect cell expression vector pFastBac1, thereby constructing a recombinant expression vector pFastBac1-gp96-plus. The recombinant plasmids were transformed into DH10Bac™ competent cells respectively, and the recombinant bacmid DNA was obtained by recombination screening.

II. Expression of gp96-Plus Protein

1. Adherent Sf9 cells were transfected with the recombinant bacmid DNA (approximately 2 μg of recombinant plasmid per 8×10⁵ Sf9 cells; during the transfection process, the transfection reagent was Cellfectin II reagent (purchased from Life technologies, Cat. No.: 10362-100)), incubated at 27° C. for 72 hours, and centrifuged, and the supernatant was P1 generation virus.

2. Sf9 cell Suspension 1 (containing 8×10⁶ Sf9 cells) was cultured at 27° C. for 1 to 5 hours to obtain adherent cultured cells; then the P1 generation virus (with dosage of 0.05 to 0.1 MOI) was added to the adherent cultured cells, incubated at 27° C. for 72 hours, and centrifuged at 4000 rpm for 5 minutes, and the supernatant was P2 generation virus.

3. The P2 generation virus (with dosage of 0.05 to 0.1 MOI) was added to Sf9 cell Suspension 2 (containing 8×10⁶ Sf9 cells), cultured at 27° C. and 100 to 120 rpm for 72 hours, and centrifuged at 4000 rpm for 5 minutes, and the supernatant was P3 generation virus.

III. Purification of gp96-Plus Protein

1. The P3 generation virus (with dosage of 0.05 MOI) was added to 300 ml of Sf9 cell Suspension 3 (containing 2 to 4×10⁶ Sf9 cells/ml), cultured at 27° C. and 100 to 120 rpm for 72-96 hours to obtain a suspension.

2. The suspension was taken, and centrifuged at 7000 rpm for 20 minutes to obtain Supernatant 1.

3. The Supernatant 1 was taken, and filtered through a 0.22 mm filter membrane to obtain a sample solution.

4. The sample solution was loaded onto a HiTrap-Q Sepharose ion exchange chromatography column (flow rate of 1 ml/min), which was then eluted with 5 ml of pH7.5, 200 mM PBS buffer (flow rate of 1 ml/min); then eluted with 10 ml of pH7.5, 300 mM PBS buffer (flow rate of 1 ml/min); and finally eluted with 3 ml of pH7.5, 600 mM PBS buffer (flow rate of 1 ml/min), the solution that passed through the column was collected and underwent ultrafiltration concentration using an ultrafiltration tube with a molecular weight cutoff of 50 KD to obtain about 1 ml of a concentrated solution.

5. The concentrated solution was loaded onto a Superdex 200 10/300GL molecular sieve chromatography column (flow rate of 0.25 mL/min), which was then eluted with pH 7.5, 150 mM PBS buffer (flow rate of 0.25 mL/min), the penetration solution at the 9^{th} to 12^{th} mL was collected and underwent further ultrafiltration using an ultrafiltration tube with a molecular weight cutoff of 50 KD to obtain a gp96-plus solution. The protein concentration of the gp96-plus solution was determined by the BCA assay, and finally the solution was aliquoted and stored at −80° C. The concentrate comprises the recombinant heat shock protein gp96-plus. The protein concentration in the protein solution was determined by the BCA assay, and finally the solution was adjusted to reach a protein concentration of 1 mg/ml, aliquoted and stored at −80° C.

### Example 4: Use of pgp96, rgp96 or gp96-Plus in Treatment of Amyotrophic Lateral Sclerosis

### I. Grouping and Immunization of Mice

### 1. Acquisition of Diseased Mice

90-Day-old mice weighing 23 to 26 g were selected, and those with limb tremor and/or limb weakness during the observation of 2 consecutive days were identified as diseased mice.

### 2. Grouping and Immunization of Mice

80 Diseased 90-day-old mice were selected, including 40 males and 40 females. The male and female mice were randomly divided into pgp96 treatment group, rgp96 treatment group, gp96-plus treatment group, and control group, and were treated as follows:

For the pgp96 treatment group: the mice were subcutaneously injected with the solution of pgp96 prepared in Example 1 every week, for a total of 8 injections, and each injection dose was 200 μg/mouse.

For the rgp96 treatment group: the mice were subcutaneously injected with the solution of rgp96 prepared in Example 2 every week, for a total of 8 injections, and each injection dose was 200 μg/mouse.

For the gp96-plus treatment group: the mice were subcutaneously injected with the solution of gp96-plus prepared in Example 3 every week, for a total of 8 injections, and each injection dose was 200 μg/mouse.

For the negative control group: the mice were subcutaneously injected with pH7.4, 0.01 mol/L PBS buffer every week, for a total of 8 injections, and each injection dose was 200 μL/mouse.

### II. Pgp96, Rgp96 and Gp96-Plus Induce the Generation of Regulatory T Cells, Reduce the Number of Inflammatory and Autoimmune Th17 Cells; Downregulate Th1 and Upregulate Th2 Immunity.

Seven days after the 3^{rd} immunization, 10 mice in each group were killed, and mouse PBMCs were isolated. The levels of Th1, Th2, Th17 and regulatory T cells (Tregs) in the mice were analyzed by flow cytometry. The regulatory T cells were CD4+CD25+FOXP3+ regulatory T cells; the Th17 cells was CD4+T cells that produced IL-17 (interleukin 17); the Th1 cells were CD4+T cells that produced IFN-γ (interferon-γ), TNFβ (tumor necrosis factor β), granulocyte-macrophage colony-stimulating factor (GM-CSF), IL-2, and lymphotoxin (LT); the Th2 cells were CD4+T cells that produced IL4, IL5, IL-9, IL-10 and IL-13. For details on the isolation and detection of regulatory T cells, please refer to Xinghui Li, et al. 2013. Induction of regulatory T cells by high-dose gp96 suppresses murine liver immune hyperactivation. PLOS One. 8(7):e68997.

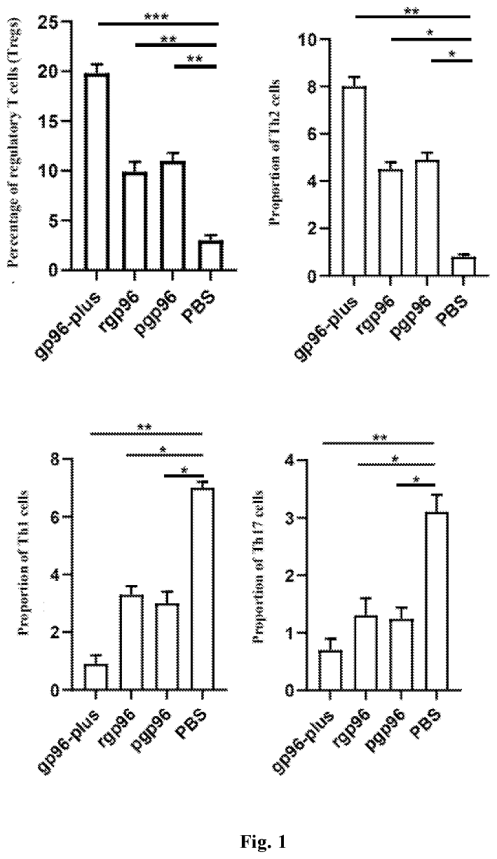

The test results were shown in FIG. 1. FIG. 1 showed the percentages of regulatory T cells (Tregs) in CD4+T cells after the mice were immunized with pgp96, rgp96 and gp96-plus proteins, respectively. The results indicated that compared with the mice of the negative control group, the levels of Treg and Th2 cells in the pgp96 treatment group, the rgp96 treatment group and the gp96-plus treatment group were significantly increased (P<0.0001), while the levels of Th1 and Th17 cells were significantly decreased. Compared to the pgp96 treatment group and the rgp96 treatment group, the gp96-plus treatment group showed a superior effect in the induction of regulatory T cells, reducing the numbers of inflammatory and autoimmune Th17 cells, and the immune function of downregulating Th1 and upregulating Th2.

III. Pgp96, Rgp96 and Gp96-Plus Reduce Reactive Oxygen Species and Oxidative Stress in Motor Neurons, Restore Function of Dysfunctional Motor Neuron Mitochondria, Reduce Denatured Proteins in Cells, Inhibit Creatine Kinase Activity, and Upregulate Creatine Level.

### 1. Determination of Reactive Oxygen Species (ROS) in Sera of Mice

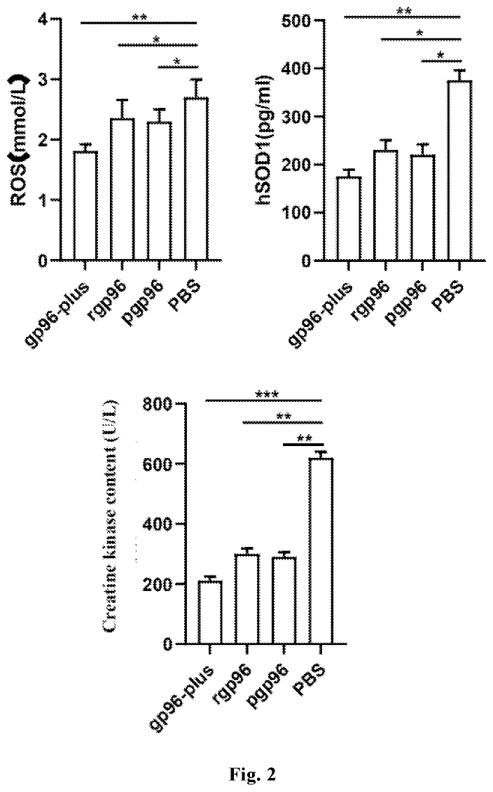

Reactive oxygen species (ROS) are important factors produced by aerobic cells during metabolism and capable of inducing oxidative stress in neurons. Studies have shown that a large amount of ROS directly attack mitochondria and cause neuronal damage. Therefore, the level of ROS can indirectly reflect the severity of free radical attack on neurons in ALS mice. The ELISA was used to detect the content of ROS in sera of mice (see the literature for specific methods). The results were shown in FIG. 2. Compared with the mice of the negative control group, the serum ROS contents of mice in the pgp96 treatment group, the rgp96 treatment group and the gp96-plus treatment group were significantly reduced (P<0.01), indicating good free radical scavenging ability, which could inhibit oxidative stress response in the mice, thereby protecting neurons.

### 2. Determination of SOD1 (Superoxide Dismutase 1) Content in Sera of Mice

ELISA kit was used to detect the changes in the expression level of hSOD1 in sera of the mice. The results were shown in FIG. 2. Compared with the mice of the negative control group, the SOD1 levels in the sera of the mice of the pgp96 treatment group, the rgp96 treatment group and the gp96-plus treatment group were significantly reduced, indicating significant decrease in expression of hSOD1 in SOD1-G93A mice, which cut off the subsequent aggregation of hSOD1 at the source, protected motor neurons, and alleviated the pathological manifestations of SOD1-G93A mice.

### 3. Detection of Creatine Kinase Content

Elevated serum creatine kinase (CK) is considered to be a marker of muscle damage. In ALS, CK levels can reflect the severity of the underlying disease process and the degree of muscle denervation. ELISA was used to detect the serum CK contents in the mice. The results were shown in FIG. 2. Compared with the mice of the negative control group, the CK contents of the mice of the pgp96 treatment group, the rgp96 treatment group and the gp96-plus treatment group were significantly reduced.

### 4. Change in Mitochondrial Membrane Potential (MMP)

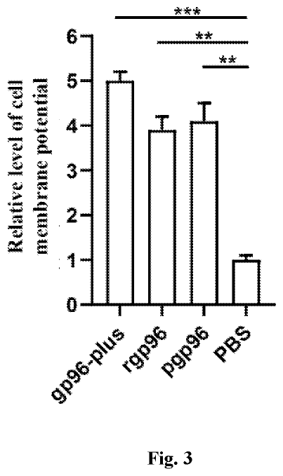

Transmembrane potential represents the mitochondrial function in living cells. Mouse bone marrow cells were isolated and the mitochondrial membrane potentials were measured by flow cytometry. The bone marrow cells were resuspended with 1 ml of 0.01M PBS solution. Rhodamine 123 dye with a final concentration of 10 ug/ml was added, the precipitate was gently suspended by pipetting, and incubated at 37° C. for 30 min in the dark. After centrifugation at 300 g for 5 min, the cells were washed twice with 1 ml of 0.01M PBS solution and immediately counted and analyzed by flow cytometry at the corresponding wavelength (Ex/Em: 488/525 nm). The results were shown in FIG. 3. Compared with the mice of the negative control group, the MMP of the mice in the pgp96 treatment group, rgp96 treatment group and gp96-plus treatment group increased significantly, indicating that the function of mitochondria in motor neurons was restored.

### IV. Pgp96, Rgp96 and Gp96-Plus Promote Production of Nerve Growth Factor, Promote Growth of Diseased Motor Nerve Axons, and Improve Axonal Transport Capacity

### 1. Tissue Sampling

Seven days after the 3^{rd} immunization, 10 mice in each group were killed and mouse specimens were obtained.

### 1) Fresh Tissue Sampling

The specific steps were as follows:

The mice were anesthetized with freshly prepared 10% chloral hydrate (1 ml/100 g) by intraperitoneal injection and then killed by cervical dislocation. After rapid soaking in 70% alcohol for 30s, they were placed in a 10 mm sterile culture dish and D-PBS was added followed by separating brain, spinal cord and muscle tissues.

### 2) Fixed Tissue Sampling

The specific steps were as follows:
a) The mice were anesthetized with freshly prepared 10% chloral hydrate (1 ml/100 g) by intraperitoneal injection.
b) After the mice were thoroughly anesthetized, they were fixed in a metal tray in a supine position with tape to fully expose the chest and abdomen. Tissue scissors were used to cut the abdominal and chest skin, diaphragm and bilateral ribs, peritoneum and other tissues from bottom to top to fully expose the heart and liver.
c) 4° C. saline solution was prepared, an intravenous puncture needle was slowly and gently inserted into the left ventricle (slight breakthrough feeling, piercing heart was avoided), the puncture needle was fixed with hemostatic forceps, the flow regulator was turned on, and it was observed that the liquid slowly dripped from a dripping funnel of infusion device, which proved that the puncture was successful and the perfusion path was formed. At the same time, ophthalmic scissors were used to cut the right atrial appendage, which was conducive to the full outflow of circulating blood. The flow regulator was adjusted to the maximum for rapid perfusion. In total, about 20 ml of saline solution was required.
d) After observing that the mouse liver was perfused to white and clear liquid flowed out of the right atrial appendage, the perfusion of saline solution was stopped, and it was switched to 4° C. 4% paraformaldehyde (prepared with 0.01M PBS) for rapid tissue fixation, and its usage amount was about 20 ml.
e) When the mouse's head, neck, limbs and tail were stiff, it indicated that the paraformaldehyde tissue fixation was completed, then the mouse was decapitated with ophthalmic scissors, and the intact mouse spinal cord tissue was carefully peeled out with a tweezer.
f) The whole mouse spinal cord tissue was soaked in 4% paraformaldehyde (prepared with 0.01M PBS) and placed in a 4° C. refrigerator overnight to continue fixation.
g) The mouse spinal cord tissue that had been fixed by soaking in paraformaldehyde overnight was soaked in 20% sucrose (0.01M PBS) solution for initial dehydration. After it sank to the bottom, 30% sucrose (0.01M PBS) solution was then used for replacement to perform dehydration again until it sank to the bottom.
h) After gradient dehydration, the mouse spinal cord tissue was taken out and absorbed with filter paper to remove surface moisture, and the mouse spinal cord tissue was divided into cervical, thoracic and lumbar segments. The spinal cord tissue segments were embedded in sequence using OCT glue, and marked separately to clearly indicate the cervical, thoracic and lumbar segments, and finally stored in a −80° C. refrigerator.

### 3) Frozen Section of Experimental Specimens

The mouse spinal cord tissue embedded with OCT glue was placed in a constant-temperature freezing microtome for slicing, and the slice thickness was set to about 12 um for subsequent immunofluorescence staining.

The frozen section operation method was as follows:
a) The mouse spinal cord tissue was taken from the −80° C. refrigerator, placed on the operating table of constant-temperature freezing microtome, and rewarmed for 30 minutes.
b) The tissue specimen was fixed in the center of the tissue specimen table using OCT glue, and quickly frozen for 2 to 3 minutes. After the OCT glue was dry, the tissue specimen table was installed on the slicer head and tightened.
c) The technical experimenter gradually trimmed the tissue plane to expose the mouse spinal cord tissue embedded therein.
d) The slice thickness of the constant-temperature freezing microtome was set to 12 um, the spinal cord tissue was cut continuously according to the coronal position, and the obtained slices were quickly adhered to anti-slip slides.
e) After the mouse spinal cord tissue specimen was sliced, the resultant slices were placed in a slice box, sealed with plastic wrap, and stored in a −80° C. refrigerator.

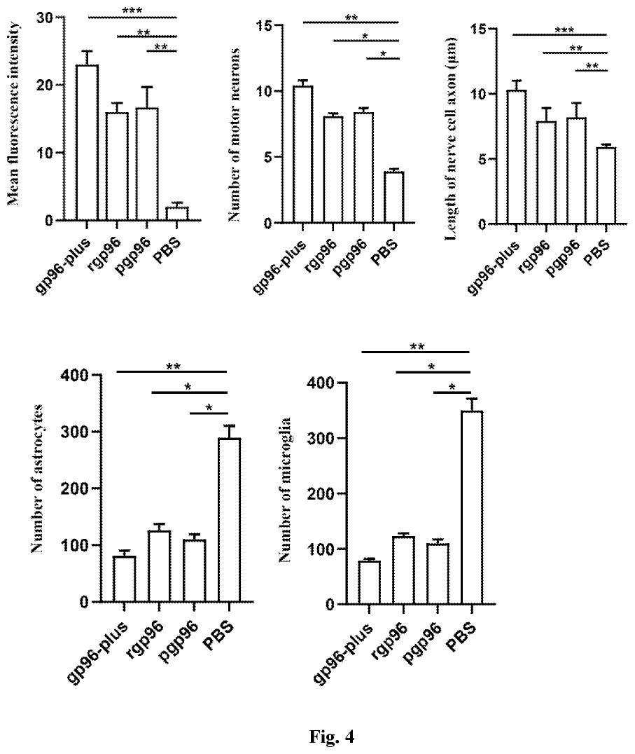

2. Neuronal growth factor (NGF) detection was performed on the mouse spinal cord tissue by immunofluorescence double staining. The experimental results were shown in FIG. 4, in which the level of NGF-positive cells in the pgp96 treatment group, the rgp96 treatment group and the gp96-plus treatment group increased significantly as compared with the negative control mice, which prevented the death of nerve cells in ALS and slowed down the progression of ALS.

### 3. Motor Neuron Detection

The spinal cord tissue of mice was immunofluorescently stained, and the experimental results were shown in FIG. 4. Compared with the mice of the negative control group, the numbers of neurons in the pgp96 treatment group, the rgp96 treatment group and the gp96-plus treatment group increased significantly, the length of nerve cell axons increased significantly, and the numbers of astrocytes and microglia decreased significantly, indicating that motor neurons were protected, and neuronal degeneration and necrosis were delayed. Among them, the gp96-plus treatment group was better than the pgp96 treatment group and rgp96 treatment group in promoting the production of nerve growth factor, and promoting the growth of diseased motor nerve axons.

### V. Evaluation of Effect of Injection of Heat Shock Protein Gp96 and Gp96-Plus on Disease Progression in Mice

The weight, survival rate, neurological function score and motor ability of hSOD1-G93A transgenic mice were observed and recorded on days 90, 100, 110, 120, 130, 140, 150, 160, 170 and 180, respectively. Time of death: when a mouse was placed in supine position, if it could not turn over to prone position within 20 seconds, it was considered dead.

### 1. Neurological Function Score

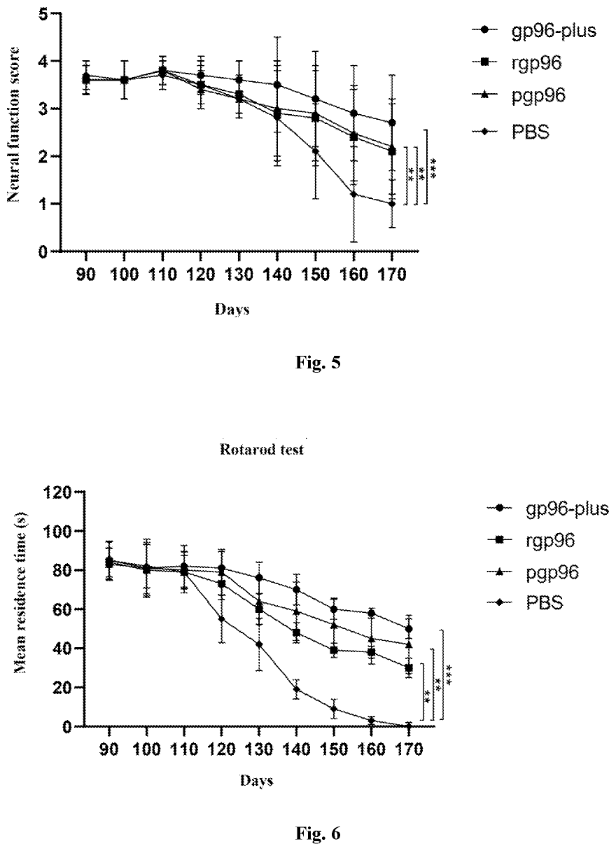

According to the neurological function scoring criteria of mice, the neurological function score of ALS mice was calculated. The average score of each group of mice was taken to obtain the average neurological function score of each group. The curve was drawn with the number of days as the horizontal axis and the score as the vertical axis. The scoring standard referred to Table 2. The results were shown in FIG. 5. As time went by, the abnormal neurological function of the mice in the negative control group gradually worsened; the neurological function of the mice in the pgp96 treatment group, the rgp96 treatment group and the gp96-plus treatment group showed significant improvement; and the improvement of the neurological function in the gp96-plus treatment group was better than that in the pgp96 treatment group and the rgp96 treatment group.

TABLE 2
Neurological function scoring criteria

| Scoring criteria | Score |
|---|---|
| Normal neurological function, no movement disorder | 4 |
| Tremor or abnormal hind foot extension when suspending mouse | 3 |
| Obvious hind limb weakness and abnormal gait in mice | 2 |
| Complete paralysis of hind limbs, crawling only on forelimbs in mice | 1 |
| Righting reflex disappeared, unable to turn over within 30 seconds when lying on | 0 |
| back in mice | |

### 2. Evaluation of Motor Ability:

### 1) Rotarod Test

Rotarod test can evaluate the coordination, strength and balance of movement. Starting from day 90, the rotarod movement of the mice was tested every 10 days, the speed was set to 12 rpm/min, and the time from the beginning to the rod falling of the mice was recorded within 5 minutes. Each experiment was repeated 3 times. The results were shown in FIG. 6. The mice in the negative control group could no longer stay on the rotating rod after 150 days of age. The mice in the pgp96 treatment group, the rgp96 treatment group and the gp96-plus treatment group stayed on the rotating rod for a significantly longer time than those in the negative control group (p<0.01). The limb strength and motor coordination ability of the mice were significantly improved.

### 2) Suspension Line Test

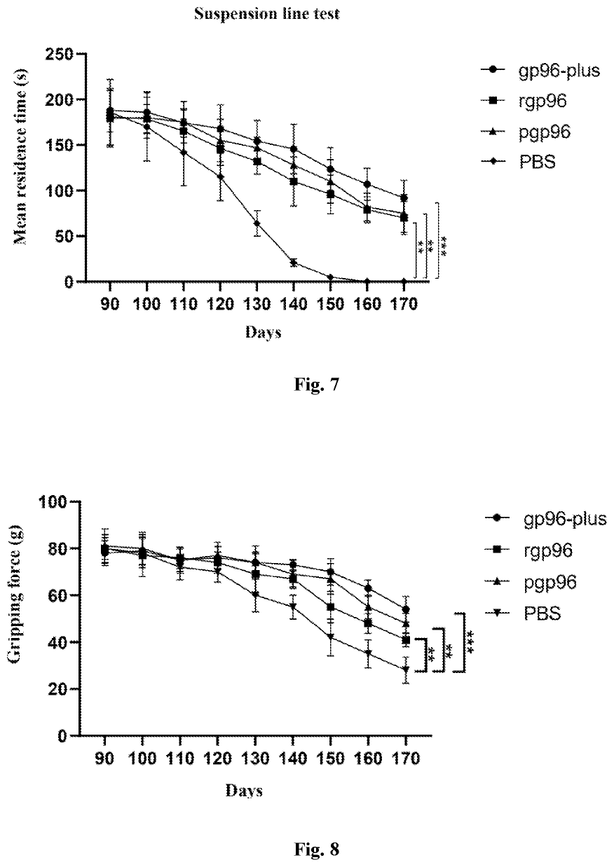

This test was mainly used for evaluating the grasping strength of mice. The mice were placed on a traditional mouse cage cover, and the cage was gently shaken to enable the mice to hold the mouse cage cover tightly, then the cage cover was quickly turned over, and the longest latency of the hind limbs leaving the cage cover was recorded. Each experiment was repeated three times and the average value was taken. The results were shown in FIG. 7. The hanging time of the mice in the negative control group showed a significant downward trend from 90 days of age, the hanging time was close to 0 after 140 days of age, indicating that the mice had a serious loss of limb strength. The limb strength of the mice in the pgp96 treatment group, the rgp96 treatment group and the gp96-plus treatment group was significantly improved, indicating that the loss of limb strength was delayed.

### 3) Hind Foot Gripping Test

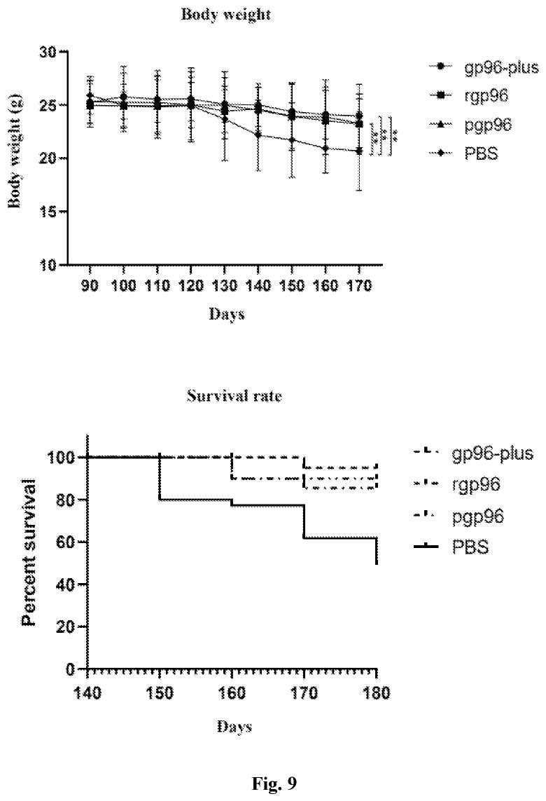

This test was mainly used for evaluating the gripping force of mice. Starting from day 90, the mice were subjected to suspension test every 10 days. The gripping force of mice in each group was tested with a mouse muscle strength tester. The mice were gently placed on the tester platform, so that their limbs were tightly grasped on the tester platform, then the tail of mouse was slightly pulled until the mouse loosened, and the tester tension reading was recorded. Each mouse was measured repeatedly 12 times, the first 5 times were discarded, and the remaining 7 times were averaged. The results were shown in FIG. 8. The maximum pulling force of hind feet of the mice in the negative control group showed a significant downward trend, indicating that the mice had a serious loss of limb strength. The limb strength of the mice in the pgp96 treatment group, the rgp96 treatment group and the gp96-plus treatment group was significantly improved, indicating that the loss of limb strength was delayed.

### 4) Statistics of Weight and Survival Rate in Mice

The weight, survival rate and death time of mice in each group were statistically analyzed. The statistical results of weight and survival rate in the mice were shown in FIG. 9. The results showed that the weight of mice in the negative control group gradually decreased from 120 days of age, while the weight loss of mice in the pgp96 treatment group, the rgp96 treatment group and the gp96-plus treatment group was significantly improved. The mice in the negative control group began to die at 150 days of age; the mice in the pgp96 treatment group began to die at 170 days of age, and the mice in the rgp96 treatment group and the gp96-plus treatment group began to die at 160 days of age. Therefore, after treatment with pgp96, rgp96, or gp96-plus, the survival time of mice was significantly prolonged. In addition, the survival time of the mice in the gp96 treatment group and the gp96-plus treatment group was higher than that in the negative control group. The above results showed that when pgp96, rgp96 or gp96-plus was used to immunize mice, it could effectively treat or alleviate ALS symptoms, and prolong the survival time of mice. The survival time of the mice in the gp96-plus treatment group was higher than that of the pgp96 treatment group and the rgp96 treatment group.

Although the specific models of the present invention have been described in detail, those skilled in the art will understand that various modifications and changes can be made to the details based on all the teachings that have been disclosed, and these changes are within the scope of protection of the present invention. All of the invention is given by the appended claims and any equivalents thereof.

1. A method for preventing and/or treating amyotrophic lateral sclerosis in a subject, comprising: administering an effective amount of a gp96 protein or variant thereof or a fusion protein to a subject in need thereof;
wherein, the variant has a sequence identity of at least 90%, such as at least 95%, at least 96%, at least 97%, at least 98%, at least 99%; or, has a substitution (preferably conservative substitution), addition or deletion of one or several (e.g., 1, 2, 3, 4, 5, 6, 7, 8 or 9) amino acids as compared to the gp96 protein, and retains the function of the gp96 protein;
the fusion protein comprises the gp96 protein or variant thereof, and an additional peptide connected to the gp96 protein or variant thereof.

2. The method according to claim 1, wherein the additional peptide is connected to the N-terminal and/or C-terminal of the gp96 protein or variant thereof, optionally via a linker (e.g., a peptide linker);
preferably, the additional peptide is connected to the N-terminal of the gp96 protein or variant thereof.

3. The method according to claim 1, wherein the additional peptide is a flexible peptide;
preferably, the additional peptide comprises one or more glycine (G).

4. The method according to claim 1, which has one or more features selected from the group consisting of:
(i) the gp96 protein comprises or consists of an amino acid sequence as set forth in SEQ ID NO: 1 or 2;
(ii) the fusion protein comprises or consists of an amino acid sequence as set forth in SEQ ID NO: 4;
(iii) the gp96 protein or variant thereof or the fusion protein may further comprise an additional protein tag, a targeting moiety or any combination thereof.

5. The method according to claim 1, which is used for one or more of the following:
(1) inducing a regulatory T cell;
(2) inhibiting the generation of a Th17 cell;
(3) inducing an increase in the number of Th2 cell;
(4) inhibiting the generation of a Th1 cell;
(5) reducing reactive oxygen species and oxidative stress in a motor neuron;
(6) reducing the expression of SOD1;
(7) restoring the function of dysfunctional mitochondria in a motor neuron;
(8) reducing denatured protein in a cell;
(9) reducing creatine kinase level and/or inhibiting creatine kinase activity, and upregulating creatine level;
(10) promoting the production of nerve growth factor;
(11) promoting the growth of a diseased motor nerve axon;
(12) improving axonal transport capacity.

6. (canceled)

7. (canceled)

8. A fusion protein, comprising a gp96 protein or variant thereof, and an additional peptide linked to the gp96 protein or variant thereof;
wherein, the variant has a sequence identity of at least 90%, such as at least 95%, at least 96%, at least 97%, at least 98%, at least 99%; or, has a substitution (preferably conservative substitution), addition or deletion of one or several (e.g., 1, 2, 3, 4, 5, 6, 7, 8 or 9) amino acids as compared to the gp96 protein, and retains the function of the gp96 protein;
the additional peptide is linked to the N-terminal and/or C-terminal of the gp96 protein or variant thereof, optionally via a linker (e.g., a peptide linker); and the additional peptide has a structure as shown in (GGGGS)ₙ₁C(GGGGS)ₙ₂, wherein the n1 and n2 are each independently selected from: 0, 1, 2, 3, 4, 5, 6, 7, 8, 9, 10.

9. The fusion protein according to claim 8, which has one or more features selected from the group consisting of:
(i) the gp96 protein comprises or consists of an amino acid sequence as set forth in SEQ ID NO: 1 or 2;
(ii) the fusion protein comprises or consists of an amino acid sequence as set forth in SEQ ID NO: 4;
(iii) the fusion protein may further comprise an additional protein tag, a targeting moiety or any combination thereof.

10. An isolated nucleic acid molecule, encoding the fusion protein according to claim 8.

11. A vector, comprising the isolated nucleic acid molecule according to claim 10; preferably, the vector is a cloning vector or an expression vector.

12. A host cell, comprising the isolated nucleic acid molecule according to claim 10 or a vector comprising the isolated nucleic acid molecule.

13. A method for preparing a fusion protein, comprising culturing the host cell according to claim 12 under a condition that allows protein expression, and recovering the fusion protein from a culture of the cultured host cell.

14. A pharmaceutical composition, comprising the fusion protein according to claim 8, an isolated nucleic acid molecule or vector or host cell comprising a nucleotide sequence encoding the fusion protein, and a pharmaceutically acceptable carrier and/or excipient;
preferably, the pharmaceutical composition optionally further comprises an additional pharmaceutically active agent;
preferably, the additional pharmaceutically active agent is a drug having the effect of treating amyotrophic lateral sclerosis.

15. (canceled)

16. A method for preventing and/or treating amyotrophic lateral sclerosis, comprising: administering an effective amount of (i) the fusion protein according to claim 8, or (ii) an isolated nucleic acid molecule or vector or host cell comprising a nucleotide sequence encoding the fusion protein, or (iii) a pharmaceutical composition comprising any of the foregoing and a pharmaceutically acceptable carrier and/or excipient, to a subject in need thereof.

17. The method according to claim 1, wherein the additional peptide has a structure as shown in (GGGGS)ₙ₁C(GGGGS)ₙ₂, wherein the n1 and n2 are each independently selected from: 0, 1, 2, 3, 4, 5, 6, 7, 8, 9, 10.

18. The method according to claim 17, wherein the n1 and n2 are not 0 at the same time.

19. The method according to claim 1, wherein the additional peptide has an amino acid sequence as set forth in SEQ ID NO: 6.

20. The fusion protein according to claim 8, wherein the n1 and n2 are not 0 at the same time.

21. The fusion protein according to claim 8, wherein the additional peptide has an amino acid sequence as set forth in SEQ ID NO: 6.

22. The method according to claim 16, which is used for one or more of the following:
(1) inducing a regulatory T cell;
(2) inhibiting the generation of a Th17 cell;
(3) inducing an increase in the number of a Th2 cell;
(4) inhibiting the generation of a Th1 cell;
(5) reducing reactive oxygen species and oxidative stress in a motor neuron;
(6) reducing the expression of SOD1;
(7) restoring the function of dysfunctional mitochondria in a motor neuron;
(8) reducing denatured protein in a cell;
(9) reducing creatine kinase level and/or inhibiting creatine kinase activity, and upregulating creatine level;
(10) promoting the production of nerve growth factor;
(11) promoting the growth of a diseased motor nerve axon;
(12) improving the transport capacity of an axon.
